(19) United States
(12) Patent Application Publication
(10) Pub. No.: US 2008/0058345 A1
(43) Pub. Date: Mar. 6, 2008

(54) Combination Therapy with Mecamylamine for the Treatment of Mood Disorders

(76) Inventors: Tony George, Toronto (CA); Kristi Sacco, Trumbull, CT (US); Jennifer Vessicchio, Milford, CT (US)

Correspondence Address: WOLF GREENFIELD & SACKS, P.C., 600 ATLANTIC AVENUE, BOSTON, MA, 02210-2206, US

(21) Appl. No.: 10/585,562

(22) PCT Filed: Jan. 4, 2005
(86) PCT No.: PCT/US05/00083

§ 371 Date: Mar. 6, 2007

Related U.S. Application Data
(60) Provisional application No. 60/534,532, filed on Jan. 6, 2004.
(60) Provisional application No. 60/627,250, filed on Nov. 12, 2004.

(51) Int. Cl. A61K 31/4439; A61K 31/135; A61K 31/343; A61P 25/24; A61K 31/496; A61K 31/44

(52) U.S. Cl. 514/254.06; 514/338; 514/343; 514/357; 514/469; 514/646; 514/789

(57) ABSTRACT

A combination therapy for treating mood disorders, including depressive disorders such as major depression, panic disorder, obsessive compulsive disorder, social anxiety disorder, seasonal affective disorder, and premenstrual dysphoria is provided herein. The combination therapy comprises administering an effettive amount of mecamylamine together with at least one agent for treatment of a mood disorder to a patient in need of treatment of the mood disorder. Pharmaceutical formulations, including packaged pharmaceutical formulations, comprising mecamylamine and an additional agent, such as an SSRI, are also provided herein.

### RELATED APPLICATIONS

This application claims the benefit of U.S. Provisional Application 60/534,532, entitled “Nicontinic Antagonist Augmentation of Antidepressants including SSRIs MAOIs, TCAs, NRI/SSRIs and others,” by Tony Peter George, filed Jan. 6, 2004, and U.S. Provisional Application 60/627250 filed Nov. 12, 2004. The entire teachings and specification of the referenced applications are incorporated herein by reference.

### FUNDING

Work described herein was funded, in whole or in part, by National Institutes of Health/National Institute of Drug Abuse Grant No. 5R01-DA-14039; National Institutes of Health/National Institute of Drug Abuse Grant No. 5R01-DA-13672; and National Institutes of Health/National Institute of Drug Abuse Grant No. 1R01-DA-15757. The United States government has certain rights in the invention.

### BACKGROUND

Mood disorders make up a class of psychiatric disorders characterized by an exaggerated mood state and include, for example, bipolar disorder and major depression. The prevalence of mood disorders is high; the lifetime prevalence of major depressive disorder (MDD) in the United States is about 15%:

Numerous therapies are available for treating patients suffering from mood disorders. However, a large percent of those treated with conventional drug therapies experience only partial relief of their symptoms or do not respond at all to such treatments. For example, Selective Serotonin Reuptake Inhibitors (SSRIs) are currently used in the treatment of MDD and are generally considered by psychiatrists and primary care physicians to be effective, well-tolerated and easily administered. However, they are associated with undesirable features, such as delayed onset of action, a level of non-responsiveness estimated to be as high as 30%, and a high incidence of sexual dysfunction.

Thus, there there is a need for pharmaceutical therapies that can be used to treat mood disorder patients who do not respond to currently available therapies, as well as for pharmaceutical therapies that improve the efficacy of currently available treatment regimens.

### SUMMARY OF THE INVENTION

As described herein, administering a (at least one) nicotinic acetylcholine receptor (nAChR) antagonist, such as an (at least one) antagonist of high affinity nAChRs, and at least one additional agent which is an agent for treating a mood disorder is a more effective method of treating a mood disorder in an individual than treatment in which an agent for treating a mood disorder is administered alone (in the absense of/not in conjunction with such an antagonist or antagonists). Administration of an antagonist of high affinity nAChR (a high affinity nAChR antagonist), such as mecamylamine or a salt thereof, with at least one additional agent (one or more additional agents) which is an agent for treating mood disorders provides a more effective method of treating mood disorders in an individual in need of such treatment than results if the one or more agents for treating mood disorders is administered in the absence of an nAChR antagonist, such as mecamylamine. Such an approach—combination or co-administration of the two types of agents—is particularly useful for treating individuals suffering from a mood disorder who do not respond to currently-available therapies and is also useful for improving the efficacy of currently-available mood disorder therapies for individuals who do respond to such therapy.

Pharmaceutical compositions which are useful for treating mood disorders and comprise an (at least one) nAChR antagonist and at least one additional agent which is an agent for treating a mood disorder are a subject of the present invention. In specific embodiments, pharmaceutical compositions of the present invention comprise an antagonist of high affinity nAChRs and at least one agent for treating a mood disorder. In specific embodiments, a pharmaceutical composition of the present invention comprises a high affinity nAChR antagonist such as mecamylamine or a salt thereof (e.g., mecamylamine hydrochloride) and at least one of the following: a selective serotonin reuptake inhibitor (SSRI), a tricyclic antidepressant (TCA), a monoamine oxidase inhibitor (MAOI), a selective norepinephrine reuptake inhibitor (SNRI) or other antidepressant. Pharmaceutical compositions of the present invention are useful to treat a wide variety of mood disorders, including, but not limited to, major depressive disorder; SSRI refractory major depressive disorder; SSRI refractory mood and anxiety disorders, such as panic disorder, post-traumatic stress disorder (PTSD), bipolar disorder, dysthymic disorder and minor depression. In embodiments in which a pharmaceutical composition of this invention comprises mecamylamine and a selective serotonin reuptake inhibitor, the latter can be, for example, fluoxetine, sertraline, citalopram, paroxetine or fluvoxamine.

Also the subject of this invention are methods of treating mood disorders. Such a method comprises administering an effective amount of an (at least one) nAChR antagonist and at least one agent which is an agent for treating a mood disorder to an individual in need of such treatment. In particular embodiments of the method, mecamylamine or another antagonist of high-affinity nAChRs and at least one agent which is an agent for treating a mood disorder is administered to an individual in need of such treatment, such as an individual suffering from a mood disorder (e.g., major depressive disorder, SSRI refractory major depressive disorder; SSRI refractory mood and anxiety disorders, such as panic disorder, post-traumatic stress disorder (PTSD), bipolar disorder, dysthymic disorder and minor depression). The treatment method can be carried out by administering an antagonist of high-affinity nAChRs and at least one agent for treating a mood disorder, such as depression, together (in a composition which comprises both the antagonist and the agent for treating depression) or individually (separately/as two separate agents). In the embodiment in which the two are administered separately, they are administered sufficiently close in time to result in the desired effect (enhanced or more effective treatment of the mood disorder than occurs when the one or more agent(s) for treating a mood disorder is administered without the antagonist of nAChRs). For example, mecamylamine, such as mecamylamine hydrochloride, and at least one agent for treating depression can be administered to an individual in need of treatment of depression. The agent for treating depression can be, for example, a SSRI, a TCA, a MAOI, a SNRI or other antidepressant. In embodiments in which a selective serotonin reuptake inhibitor is administered, it can be, for example, fluoxetine, sertraline, citalopram, paroxetine or fluvoxamine.

In certain embodiments disclosed herein, the active agent is a chemical compound known to be effective for treating the mood disorder. For example, in one embodiment of the method of treating the mood disorder major depression, an effective amount of mecamylamine is administered with at least one additional agent, such as a SSRI (e.g., sertraline hydrochloride, fluoxetine, paroxetine, citalopram or fluvoxamine), which is known to be effective for treating depression.

Packaged pharmaceutical formulations and pharmaceutical kits comprising an antagonist of nAChRs, such as an antagonist of high affinity nAChRs (e.g., mecamylamine or a salt thereof), and at least one agent for treating a mood disorder, such as an agent for treating depression, are described. Extended release forms of mecamylamine useful in the pharmaceutical compositions, packaged formulations, and kits described herein are also disclosed.

These and other embodiments of the invention, as well as additional inventive features, will be apparent from the description of the invention provided herein.

### BRIEF DESCRIPTION OF THE DRAWINGS

FIG. 1 is a graph showing the number of responders in each study medication group (MEC=mecamylamine plus antidepressant; Placebo=placebo plus antidepressant).

FIGS. 2A and 2B are graphic representations showing results of assessments using the Hamilton Depression Scale-17 and Hamilton Depression Scale-21, respectively.

FIG. 3 is a graph showing results of assessments using the Montgomery Ashburg Depression Scale.

FIG. 4 is a graph showing results of assessments using the Beck Depression Inventory.

### DETAILED DESCRIPTION OF THE INVENTION

### Terminology

Prior to setting forth the invention in detail, it may be helpful to provide definitions of certain terms to be used herein. Compounds of the present invention are described using standard nomenclature. Unless defined otherwise, all technical and scientific terms used herein have the same meaning as is commonly understood by one of skill in the art to which this invention belongs.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

Agents may contain one or more asymmetric elements such as stereogenic centers or stereogenic axes e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, including mecamylamine, it should be understood that all of the optical isomers and mixtures thereof are encompassed. In addition, compounds with carbon-carbon double bonds may occur in Z- and E-forms; all isomeric forms of the compounds are included in the present invention. In these situations, the single enantiomers (optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Unless otherwise specified, or clearly indicated by the text, “mecamylamine” includes both the free base of mecamylamine, (Bicyclo(2.2.1)heptan-2-amine, N,2,3,3-tetramethyl-), and all pharmaceutically acceptable salts of this compound. The preferred mecamylamine salt is mecamylamine hydrochloride. The term “mecamylamine or its salts” indicates the pharmaceutically acceptable salts of mecamylamine.

The term “pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

An “effective amount” of an agent or of a combination of an nAChR antagonistpharmaceutical formulation is an amount sufficient to provide an observable improvement over the baseline clinically observable signs and symptoms of the disorder.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “releasable form” is meant to include instant release, immediate-release, controlled-release, and sustained-release forms.

By “instant-release” is meant a dosage form designed to ensure rapid dissolution of the active agent by modifying the normal crystal form of the active agent to obtain a more rapid dissolution.

By “immediate-release”, it is meant a conventional or non-modified release form in which greater then or equal to about 50% or more preferably about 75% of mecamylamine or other active agent is released within two hours of administration, preferably within one hour of administration.

By “controlled-release” it is meant a dosage form in which the meamylamine or other active agent release is controlled or modified over a period of time. Controlled can mean, for example, sustained, delayed or pulsed-release at a particular time. Alternatively, controlled can mean that the mecaymylamine or other active agent release is extended for longer than it would be in an immediate-release dosage (e.g., over one or several hours.

By “sustained-release” or “extended-release” is meant to include the release of mecamylamine or other active agent at such a rate that blood (e.g., plasma) levels are maintained within a therapeutic range but below toxic levels for at least about 8 hours, preferably at least about 12 hours after administration at steady-state. The term “steady-state” means that a plasma level for a given active agent, such as mecamylamine or other active agent, has been achieved and which is maintained with subsequent doses of the drug at a level which is at or above the minimum effective therapeutic level and is below the minimum toxic plasma level for a given active agent.

By “water-soluble” agent is meant an agent, including mecamylamine hydrochloride or other active agent hydrochloride, and active agents that may be used in combination with mecamylamine or other active agent that are at least slightly water-soluble (for example, about 1 to about 10 mg/ml at 25° C.). Preferably, all active agents are moderately water-soluble (for example, less than about 100 mg/ml at 25° C.), or highly water-soluble (for example, greater than about 100 mg/ml at 25° C.).

By “water-insoluble” or “poorly soluble” active agent, it is meant an agent having a water solubility of less than 1 mg/ml, and in some cases even less than 0.1 mg/ml.

### Methods of Treatment

Disclosed herein is a method of treating a mood disorder, such as depression, in an individual by administering an nAChR antagonist, such as a high affinity nAChR (central nervous system or brain nicotonic receptor(s)) antagonist (e.g., mecamylamine), in combination with at least one additional agent which is an agent for treating a mood disorder.

The methods of the present invention are useful for the treatment of a variety of mood disorders. Mood disorders, also called affective disorders, are mental disorders characterized by persistant or episodic exaggeration of a mood state.
Mood disorders include, but are not limited to, the following disorders:
Mood Episodes, e.g. Major Depressive Episode, Hypomanic Episode, Manic Episode, and Mixed Episode;
Depressive Disorders: e.g., Dysthymic Disorder, Major Depressive Disorder: single episode; Major Depressive Disorder: recurrent, and Depressive Disorder NOS, depressive neurosis, depressive reaction, postpartum depression, premenstrual syndrome, and neurotic depression;
Bipolar Disorders: e.g. Bipolar I Disorder, Bipolar II Disorder, and Cyclothymic Disorder; and
Mood Disorders due to a general medical condition: e.g. with Depressive, Manic, or Mixed Features. The method of the present invention is also useful to treat anxiety disorders, such as obsessive-compulsive disorder, social phobias, and post-traumatic stress disorder.

Methods of treating a mood disorder by administering an effective amount of mecamylamine, in combination with an effective amount of at least one agent for the treatment of the mood disorder, to an individual having a mood disorder, such as major depressive disorder, dysthymic disorder, depressive disorder NOS, or bipolar disorder, are provided herein.

A method of treating major depressive disorder, by administering an effective amount of mecamylamine, in combination with an effective amount of at least one agent which is an agent for the treatment of major depressive disorder, to an individual in need thereof is particularly provided herein.

The agent administered with mecamylamine, referred to herein as an additional agent which is an agent for the treatment of a mood disorder or mood disorders, is typically a chemical compound known to be useful for treating mood disorders. Thus, included herein are methods of treating mood disorders comprising administering mecamylamine in combination with one or more of the following: a tricyclic antidepressant, a monoamine oxidase inhibitor, a selective serotonin reuptake inhibitor, a selective norepinephrine reuptake inhibitor, a norepinephrine dopamine reuptake inhibitor, or any drug or drug combination that blocks nAchr with low potency.

Tricyclic antidepressants include, but are not limited to, doxepin, amitriptyline, amoxapine, clomipramine, desipramine, doxepin, imipramine, maprotiline, nortriptyline, protriptyline, and trimipramine maleate.

Monoamine oxidase inhibitors include, but are not limited to, isocarboxazid, phenelzine, and tranylcypromine.

Selective serotonin reuptake inhibitors, include, but are not limited to, citalopram, clovoxamine, escitalopram, femoxetine, flesinoxan, fluoxetine, fluvoxamine, paroxetine, setraline, trazodone, and zimeldine.

Selective norepinephrine reuptake inhibitors include, but are not limited to, atomoxetine, venlafaxine and duloxetine.

Norepinephrine dopamine reuptake inhibitors include, but are not limited to bupropion.

Particularly provided herein is a method of treating a mood disorder by administering an effective amount of mecamylamine, in combination with an effective amount of at least one additional agent for treating a mood disorder, to an individual having a mood disorder, wherein the additional agent is a selective serotonin reuptake inhibitor, or a selective norepinephrine reuptake inhibitor.

Methods of treatment described herein include administering mecamylamine as a racemic mixture. In certain embodiments, exo-S-mecamylamine which is substantially free of the exo-R-enantiomer (e.g., 95% or more of the S form) is administered.

### Dosages

The optimal dose of AChR antagonist and of additional agent(s) for treatment of a mood disorder can be determined empirically for each individual using known methods and will depend upon a variety of factors, including the activity of the agents; the age, body weight, general health, gender and diet of the individual; the time and route of administration; and other medications the individual is taking. Optimal dosages may be established using routine testing and procedures that are well known in the art.

Typically, between about 2.5 mg/dy to about 10 mg/dy nAChR antagonist, such as about 2.5 mg/dy to about 10 mg/dy high affinity nAChR antagonist (e.g., mecamylamine) will be administered to an individual in combination with the additional agent(s) for treatment of a mood disorder. Smaller doses can be administered as appropriate (e.g., to children, the elderly, individuals with other medical conditions). For example, less than 2.5 mg/dy nAChR antagonist (e.g., high affinity nAChR antagonist) can be administered when a smaller dose is needed. The daily dose will vary from individual to individual and from time to time for a given individual (e.g., as daily dose is adjusted with the individual's changing mental states or general health). Smaller doses are likely to be used in pediatric individuals. In general a dose which results in an intake of approximately 0.01 mg/kg body weight to approximately 0.02 mg/kg body weight will be appropriate. This range could be from 0.01 mg/kg body weight to about 0.2 mg/kg body weight.

The amount of mecamylamine or additional agent that may be combined with the carrier materials to produce a single dosage form will vary depending upon the individual treated and the particular mode of administration. Dosage unit forms will generally contain from about 2.5 to about 15 mg mecamylamine, from about 2.5 mg to about 5 mg mecamylamine, or from about 2.5 mg to about 10 mg mecamylamine and an effective amount of additional agent or agents, such as from about 10 mg to about 80 mg of the additional agent(s). In some cases, such as single dosage forms for pediatric use, a single dosage unit can contain smaller quantities (e.g., less than 2.5 mg/dy) of the nAChR antagonist (e.g., mecamylamine) and/or the additional agent(s). In some embodiments the dosage unit forms containing mecamylamine and at least one additional agent will contain the amount of the additional agent typically administered when the additional agent is administered alone.

For example, mecamylamine can be included in smaller amounts if needed (e.g., less than 2.5 mg), such as for pediatric use.

Frequency of dosage may vary depending on the compound used and the particular condition to be treated or prevented. In general, for treatment of most mood disorders, a dosage regimen of 4 times daily or less is preferred. For the treatment of depressive disorders, including major depression, a dosage regimen of 1 or 2 times daily is particularly preferred. In certain embodiments, administration at meal times is preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

### Pharmaceutical Formulations

Provided herein are pharmaceutical formulations comprising mecamylamine or a salt thereof and at least one agent for the treatment of a mood disorder. The pharmaceutical formulations may additionally comprise a carrier or excipient, stabilizer, flavoring agent, and/or coloring agent.

Provided herein are pharmaceutical formulations comprising mecamylamine and an additional agent which can be, for example, a tricyclic antidepressant, a monoamine oxidase inhibitor, a selective serotonin reuptake inhibitor, a selective norepinephrine reuptake inhibitor, a norepinephrine dopamine reuptake inhibitor, or any drug or drug combination that blocks nAChR with low potency. In specific embodiments, the additional active agent is a selective serotonin reuptake inhibitor. In such embodiments the selective serotonin reuptake inhibitor can be, for example, citalopram, clovoxamine, escitalopram, femoxetine, flesinoxan, fluoxetine, fluvoxamine, paroxetine, setraline, trazodone, or zimeldine. In other embodiments it is preferred that the additional agent is a selective norepinephrine reuptake inhibitor.

Pharmaceutical formulation comprising mecamylamine and at least one additional agent may be taken orally in the form of liquid, syrup, tablet, capsule, powder, sprinkle, chewtab, or dissolvable disc. Alternatively, pharmaceutical formulations of the present invention can be administered intravenously or transdermally.

### Dosage Forms: Release Properties

Mecamylamine is typically administered 2-3 times daily, while many of the drugs preferred as additional agents in the combination formulations provided herein are administered once a day or less. A combination formulation (mecamylamine or other nAChR antagonist in combination with an agent for treating a mood disorder) can be formulated in such a way that it is not necessary to administer mecamylamine separately between doses of the combination formulation. If the additional agent is administered once daily when given alone, the combination formulation containing mecamylamine and the additional agent can also be formulated for once daily administration. In certain embodiments the release properties of mecamylamine are modified to achieve this result.

The dosage forms comprising mecamylamine can be characterized by the release properties of the formulation. The dosage forms can be immediate or modified release dosage forms in which the rate of mecamylamine release in the blood stream is regulated. Sustained release formulations can be used to provide release over a period of time (e.g. one or more hours, several days or longer). Preferably, the sustained-release form avoids “dose dumping,” the production of a rapid rise and in the blood or plasma concentration of active agent, upon oral administration. The sustained-release oral dosage form can be formulated to provide for an increased duration of therapeutic action permitting effective once-daily dosing. Generally in a sustained-release dosage form the active agent release extends longer e.g., by several hours, than active agent release from the immediate-release dosage form.

A sustained-release dosage form generally comprises a release-retarding material. The release-retarding material can be, for example, in the form of a matrix or a coating. An agent in sustained-release form may be, for example, a particle of agent (e.g., nAChR antagonist, additional agent for treating a mood disorder) that is combined with a release-retarding material. The release-retarding material is a material that permits release of active agent at a sustained rate in an aqueous medium. The release-retarding material can be selectively chosen so as to achieve, in combination with the other stated properties, a desired in vitro release rate. A wide variety of materials useful to produce a sustained release form is known to those of skill in the art. They may be in the form, for example, of a hydrophilic polymer, a hydrophobic polymer, a combination of hydrophilic and hydrophobic polymer and can be, for example, surfaces (beads, flat surfaces) onto which an agent is coated and/or materials into which an agent is incorporated or embedded (e.g., spheres, films or other appropriate embodiments or shape).

### Formulations

The combination pharmaceutical formulations provided herein may be formulated by a variety of methods apparent to those of skill in the art of pharmaceutical formulation. The various release properties described above may be achieved in a variety of different ways. Suitable formulations include, for example, tablets, capsules, press coat formulations, easily administered formulations.

The dosage form can be prepared by various conventional mixing, comminution and fabrication techniques readily apparent to those skilled in the chemistry of drug formulations. Examples of such techniques are as follows:

(1) Direct compression, using appropriate punches and dies; the punches and dies are fitted to a suitable rotary tableting press;

(2) Injection or compression molding using suitable molds fitted to a compression unit

(3) Granulation followed by compression; and

(4) Extrusion in the form of a paste, into a mold or to an extrudate to be cut into lengths.
When particles are made by direct compression, the addition of lubricants may be helpful and sometimes important to promote powder flow and to prevent capping of the particle (breaking off of a portion of the particle) when the pressure is relieved. Useful lubricants are magnesium stearate (in a concentration of from 0.25% to 3% by weight, preferably less than 1% by weight, in the powder mix), and hydrogenated vegetable oil (preferably hydrogenated and refined triglycerides of stearic and palmitic acids at about 1% to 5% by weight, most preferably about 2% by weight. Additional excipients may be added to enhance powder flowability and reduce adherence

### Preparation of Mecamylamine Containing Subunits

Mecamylamine and/or an additional agent and any optional additives may be prepared in a variety of ways, for example as subunits. Pellets comprising an active agent can be prepared, for example, by a melt pelletization technique. In this technique, the active agent in finely divided form is combined with a binder and other optional inert ingredients, and thereafter the mixture is pelletized, e.g., by mechanically working the mixture in a high shear mixer to form the pellets (e.g., pellets, granules, spheres, beads, etc., collectively referred to herein as “pellets”). Thereafter, the pellets can be sieved in order to obtain pellets of the requisite size. The binder material may also be in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like, and combinations comprising one or more of the foregoing binders.

Oral dosage forms may be prepared to include an effective amount of melt-extruded subunits containing mecamylamine and an additional agent in the form of multiparticles within a capsule. For example, a plurality of the melt-extruded muliparticulates can be placed in a gelatin capsule in an amount sufficient to provide an effective release dose when ingested and contacting by gastric fluid.

Subunits, e.g., in the form of multiparticulates, can be compressed into an oral tablet using conventional tableting equipment using standard techniques. The tablet formulation may include excipients such as, for example, an inert diluent such as lactose, granulating and disintegrating agents such as cornstarch, binding agents such as starch, and lubricating agents such as magnesium stearate.

Alternatively, the subunits containing mecamylamine and/or an additional active agent are added during the extrusion process and the extrudate can be shaped into tablets by methods know in the art. The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates can be any geometrical shape within this size range. Alternatively, the extrudate can simply be cut into desired lengths and divided into unit doses of active agent without the need of a spheronization step.

The melt-extruded dosage forms can further include combinations of melt-extruded multiparticulates containing one or more of the therapeutically active agents before being encapsulated. Furthermore, the dosage forms can also include an amount of mecamylamine formulated for immediate-release for prompt therapeutic effect. Mecamylamine formulated for immediate-release can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

A melt-extruded material may be prepared without the inclusion of subunits containing active agent, which are added thereafter to the extrudate. The mixture is then tableted in order to provide release of mecamylamine and/or additional active agent. Such formulations can be particularly advantageous, for example, when an active agent included in the formulation is sensitive to temperatures needed for softening the hydrophobic material and/or the retardant material.

The oral dosage form containing active agent may be in the form of micro-tablets enclosed inside a capsule, e.g. a gelatin capsule. For this, a gelatin capsule as is employed in pharmaceutical formulations can be used, such as the hard gelatin capsule known as CAPSUGEL®, available from Pfizer.

### Particles

Many of the oral dosage forms described herein contain mecamylamine and/or additional agent in the form of particles. Such particles may be compressed into a tablet, present in a core element of a coated dosage form, such as a taste masked dosage form, a press coated dosage form, or an enteric coated dosage form, or may be contained in a capsule, osmotic pump dosage form, or other dosage form.

### Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Particular embodiments provided herein include capsules or tables comprising mecamylamine hydrochloride and antidepressant, such as SSRI or active SNRI. In certain embodiments, they include from about 2.5 mg to about 15.0 mg, from about 2.5 to about 10.0 mg, or from about 2.5 mg to about 5 mg mecamylamine and one or more antidepressant. They can include smaller quantities (e.g., less than 2.5 mg) mecamylamine (e.g., for pediatric use). As the additional agent, they can comprise, for example, 10 mg to 40 mg fluoxetine hydrochloride, from 10 mg to 60 mg atomoxetine HCl, from 37.5 mg to 150 mg venlafaxine hydrochloride, from 10 mg to 40 mg paroxetine HCl, or from 20 mg to 100 mg sertraline hydrochloride.

### Mecamylamine-Containing Pellets in Capsules

Oral dosage forms may be prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid.

### Mecamylamine-Containing Tablets in Capsules

The composition may be in the form of micro-tablets enclosed inside a capsule, e.g. a gelatin capsule. For this, a gelatin capsule employed in the pharmaceutical formulation field can be used, such as the CAPSUGEL hard gelatin capsule, available from Pfizer.

### Press Coat Formulations

A press coat oral dosage form of mecamylamine and at least one additional active agent comprises a core composition and a coating composition press-coated on the core. The core composition comprises a waxy material and active agent and the coating composition comprises a hydrophilic polymer and optionally active agent or a salt thereon. Preferably the mecamylamine is in the form of mecamylamine hydrochloride.

The core composition of the press coat dosage from comprises a waxy material. The waxy material can be a hydrophobic waxy material to provide controlled-release of mecamylamine. In pharmaceutical products, for example, such waxy materials may be, for example, carnauba wax, tribehenin, fatty alcohols (particularly those having 12-241 carbon atoms, such as lauryl alcohol, myristyl alcohol, stearyl alcohol, palmityl alcohol, etc.), fatty acids (particularly those having 12-24 carbon atoms, such as lauric acid, myristic acid, stearic acid, palmitic acid, etc), polyethylenes, castor wax, C₁₆₋₃₀ fatty acid triglycerides, beeswax, and combinations comprising one or more of the foregoing waxes.

The coating composition comprises a hydrophilic polymer. The hydrophilic polymer can provide for controlled-release of active agent. The hydrophilic polymer providing controlled-release may be a film-forming polymer, such as a hydrophilic cellulose polymer. Such a hydrophilic cellulose polymer may be hydroxyalkyl cellulose polymer, for example hydroxyethylcellulose (HEC), hydroxypropyl cellulose (HPC), hydroxypropylmethylcellulose (HPMC), hydroxypropylethylcellulose (HPEC), hydroxypropylpropylcellulose (HPPC), hydroxypropylbutylcellulose (HPBC), and combinations comprising one or more of the foregoing polymers.

Both the core composition and the coating composition may further include a filler, such as a water insoluble filler, water soluble filler, and mixtures thereof.

Optional excipients can also be present in the core composition and the coating composition, including lubricants (such as talc and magnesium stearate), glidants (such as fumed or colloidal silica), pH modifiers (such as acids, bases and buffer systems), pharmaceutically useful processing aids, and combinations comprising one or more of the foregoing excipients. Excipients in the coating composition can be the same or different as those in the core composition.

In formation of the dosage form, the core composition can be press-coated with the press-coat composition coating formulation to form a tablet The tablet can be further coated with optional additional coatings. The additional coatings can be pH-dependent or pH-independent, aesthetic or functional, and can include active agent in immediate or controlled-release. The additional coating may, for example, include an immediate-release dosage form of mecamylamine hydrochloride.

In forming the dosage form, the core composition components (active agent, wax, and optional excipients) are blended together and compressed into suitable cores. The blending can take place in a suitable order of addition. The cores may be blended by starting with the smallest volume component and then successively adding the larger volume components. Another process is to melt the wax and to blend active agent and optional excipients into the melted wax. Alternatively, active agent, wax and optional excipients can be blended together and then subjected to a temperature at which the wax will melt. Once cooled, the solidified mass can be milled into granules for compaction into cores.

The press coat formulations can be press-coated tablets containing from about 2.5 mg to about 15 mg mecamylamine (particularly in the form of mecamylamine hydrochloride). In one example, the press coat combination formulation comprises sertraline hydrochloride equivalent to 25 mg in an immediate-release coating composition and 5 mg mecamylamine between the core composition and the coating composition

Thus an embodiment of the invention pertains to a press-coat dosage form comprising a core composition comprising an active agent, a waxy material; and a coating composition comprising a hydrophilic polymer, wherein the coating composition is press-coated onto the core composition.

The invention also pertains to a press-coat dosage form comprising a core composition comprising active agent, preferably mecamylamine hydrochloride, a waxy material; and a coating composition comprising a hydrophilic polymer, wherein the coating composition which also contains at least one additional active agent, is press-coated onto the core.

In certain embodiments the waxy material of the press-coat dosage form core is carnauba wax, tribehenin, fatty alcohols, lauryl alcohol, myristyl alcohol, stearyl alcohol, palmityl alcohol, fatty acids, lauric acid, myristic acid, stearic acid, palmitic acid, polyethylenes, castor wax, C₁₆₋₃₀ fatty acid triglycerides, beeswax, or any combination thereof. In some embodiments of the invention the hydrophilic polymer in the coating composition of the active agent press-coat dosage form comprises a hydrophilic cellulose polymer.

An embodiment of the invention pertains to a combination press-coat dosage comprising a core composition comprising an active agent, which is mecamylamine hydrochloride and wherein the hydrophilic cellulose polymer is hydroxypropylmethyl cellulose (HPMC).

Another embodiment of the invention pertains to a combination press-coat dosage form comprising a core composition comprising an active agent, carnauba wax; and a coating composition comprising active agent and hydroxypropylmethyl cellulose (HPMC), wherein the coating composition is press-coated onto the core.

Yet another embodiment of the invention pertains to a combination press-coat dosage form comprising a core composition comprising active agent and carnauba wax, a coating composition comprising active agent and hydroxypropylmethyl cellulose (HPMC), wherein the coating composition is press-coated onto the core, and an additional coating composition comprising active agent. In some embodiments of the invention the additional coating composition is an immediate-release coating composition.

### Easily Administered Dosage Forms

The invention provides easily administerable dosage forms for administration to patients who have difficulty swallowing, to reduce the risk of choking upon administration, and to improve patient compliance. Such dosage forms are particularly useful for administration to elderly and juvenile patients. The invention provides, for example, sprinkle dosage forms, liquid formulations, taste-masked liquid dosage forms and fast-dissolve dosage forms.

### Chewable Tablets

Another solid dosage form is a chewable tablet containing mecamylamine and at least one additional agent. A chewable tablet comprises a chewable base and optionally a sweetener. The chewable base can comprise an excipient such as, for example, mannitol, sorbitol, lactose, or a combination comprising one or more of the foregoing excipients. The optional sweetener used in the chewable dosage form may be, for example, digestible sugars, sucrose, liquid glucose, sorbitol, dextrose, isomalt, liquid maltitol, aspartame, lactose, and combinations comprising one ore more of the foregoing sweeteners. In certain cases, the chewable base and the sweetener may be the same component.

The chewable dosage form may additionally contain preservatives, agents that prevent adhesion to oral cavity and crystallization of sugars, flavoring agents, souring agents, coloring agents, and combinations comprising one or more of the foregoing agents. Glycerin, lecithin, hydrogenated palm oil or glyceryl monostearate may be used as a protecting agent of crystallization of the sugars in an amount of about 0.04 to about 2.0 weight % of the total weight of the ingredients, to prevent adhesion to oral cavity and improve the soft property of the products. Additionally, isomalt or liquid maltitol may be used to enhance the chewing properties of the chewable dosage form.

### Fast Dissolving Dosage Forms

Another combination oral dosage form is a non-chewable, fast dissolving dosage form of mecamylamine and at least on additional active agent. These dosage forms can be made by methods known to those of ordinary skill in the art of pharmaceutical formulations. For example, Cima Labs has produced oral dosage forms including microparticles and effervescents which rapidly disintegrate in the mouth and provide adequate taste-masking. Cima Labs has also produced a rapidly dissolving dosage form containing active agent and a matrix that includes a nondirect compression filler and a lubricant. Zydis (ZYPREXA®) is produced by Eli Lilly as a rapidly dissolvable, freeze-dried, sugar matrix formulated as a rapidly dissolving tablet. Fast-dissolving dosage forms are disclosed in U.S. Pat. No. 5,178,878 and U.S. Pat. No. 6,221,392, which are hereby incorporated by reference for their teachings regarding fast-dissolve dosage forms.

### Optional Additional Additives for Mecamylamine Combination Formulations Excipients

Excipients useful in the combination formulations include inert substances used as a diluent or vehicle for the nAChR antagonist and/or the additional agent(s).

Excipients may be added to facilitate manufacture, enhance stability, control release, enhance product characteristics, enhance bioavailability, enhance patient acceptability, etc. Pharmaceutical excipients include binders, disintegrants, lubricants, glidants, compression aids, colors, sweeteners, preservatives, suspending agents, dispersing agents, film formers, flavors, printing inks, etc. Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders. Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose, e.g. Nymcel ZSB10®; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200®; formalin-casein, e.g. Plas-Vita®, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide.

### Fillers

Formulations can also contain a filler, such as a water insoluble filler, water soluble filler, and combinations thereof. The filler may be a water insoluble filler, such as silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, preferably lactose, glucose, fructose, sucrose, mannose, dextrose, galactose, the corresponding sugar alcohols and other sugar alcohols, such as mannitol, sorbitol, xylitol, and combinations comprising one or more of the foregoing fillers.

### Coatings

The formulations described herein may be coated with a functional or non-functional coating. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol poly (ethylene oxide), poly (ethylene terephthalate), poly(vinyl alcohol), poly(vinyl isobutyl ether), poly(vinyl acetate), poly (vinyl chloride), polyvinyl pyrrolidone, and combinations comprising one or more of the foregoing polymers.

In applications such as taste-masking, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and the like, and combinations comprising one or more of the foregoing polymers.

In controlled-release applications, for example, the coating can be a hydrophobic polymer that modifies the release properties of active agent from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 to about 50 percent by weight of the polymer. Concentrations of the plasticizer, however, can be determined by routine experimentation.

### Packaged Formulations

Packaged pharmaceutical formulations are included herein. Such packaged formulations include a pharmaceutical formulation comprising a nAChR antagonist (e.g., a hight affinity nAChR antagonist such as mecamylamine) and an additional agent for treating a mood disorder a container and instructions for using the formulation to treat an animal (typically a human patient) suffering from a mood disorder.

In certain embodiments the packaged pharmaceutical formulation contains mecamylamine hydrochloride dosage forms and separate dosage form is comprising at least one additional agent, such as a dosage form comprising an SSRI, in a container with instruction for administering the dosage form is on a fixed schedule.

Because mecamylamine is generally adminsister 2-3 times daily and many of the active agents useful for treating mood disorders are administered once/daily or less, in one embodiment the package pharmaceutical formation contains a combination pharmaceutical formulation comprising mecamylamine hydrochloride and at least one additional active agent in a single dosage form and separate dosage forms comprising mecamylamine hydrochloride, an no additional active agent, in a container, with instructions for administering the dosage forms on a fixed schedule.

The invention includes providing prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the compounds of the invention can be administered alone, as mixtures, or in combination with other active agents.

### EXAMPLES

The following examples further illustrate the invention but are not intended to limiting its scope in any way.

### Example 1

### Assessment of Combination Therapy Using Nicotine Receptor Antagonist

The treatment of depressive symptoms in individuals with major depression who are partial responders (based on HAM-D scores) to selective serotonin reuptake inhibitors (e.g., fluoxetine, sertraline, citalopram, paroxetine, fluvoxamine) was assessed.

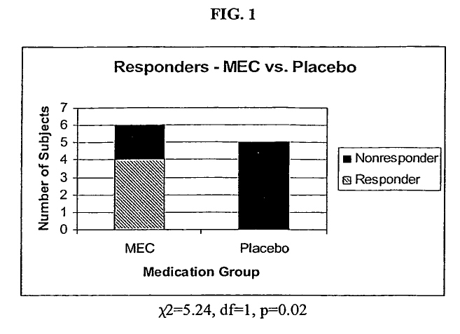

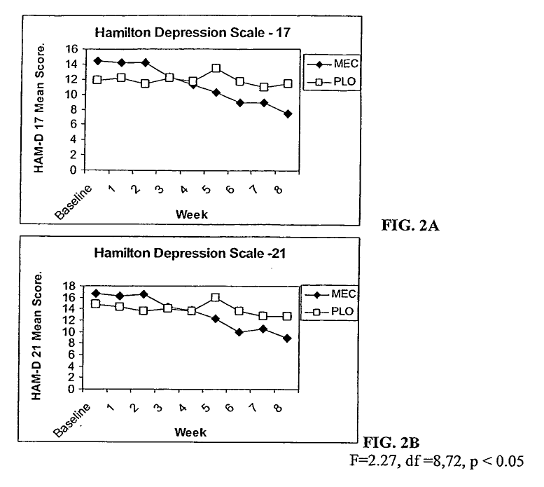

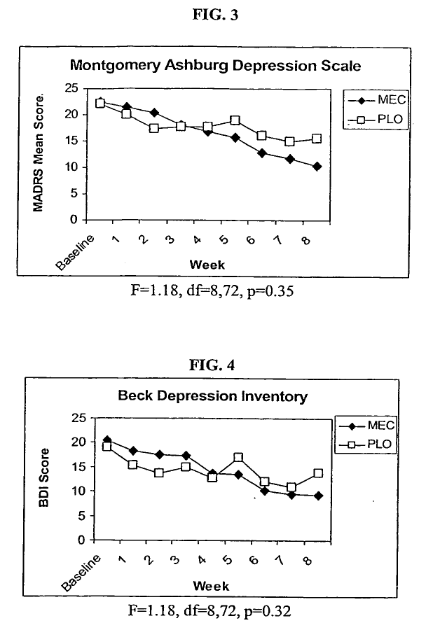

Subjects (n=13) were randomly assigned to one of two groups, designated mecamylamine or MEC and Placebo, respectively, for an 8-week clinical trial. Individuals in the MEC group received mecamylamine plus an antidepressant and those in the placebo group received placebo plus an antidepressant Eleven subjects (n=6 randomized to MEC, n=5 to placebo) have completed this 8-week clinical trial and results are presented for those individuals. Two subjects are currently enrolled in the protocol. No clinical or demographic differences have been found between subjects randomized to MEC versus Placebo (Table). Four out of six subjects assigned to active MEC were classified as responders at the end of the 8-week trial, as assessed by a 50% reduction in HAM-D scores, compared to 0/5 subjects assigned to Placebo (χ2=5.24, df=1, p=0.02) (FIG. 1). There were no significant differences between the MEC and Placebo groups on any adverse events, although the two most commonly reported were constipation (MEC 5/6), compared to (placebo 3/5) (χ2=2.5, df=1, p=0.11), and report of decreased libido (4/5 MEC) versus (1/5 placebo) (χ2=3.6, df=1, p=0.06). These preliminary results support our primary hypothesis that central nAChR antagonism augments SSRI-treated refractory major depression.

TABLE
Mecamylamine/Depression Baseline Demographic
and Clinical Characteristics by Study Medication
Status (N = 11 subjects completed)

| | | Placebo | |
|---|---|---|---|
| | MEC (n = 6) | (n = 5) | p-value |
| Age | 46.0 ± 6.6 | 45.4 ± 13.8 | p = 0.93 |
| Race | 5W/1AA | 4W/1AA | p = 0.89 |
| Gender | 1M/5F | 1M/4F | p = 0.89 |
| Years of Education | 14.6 ± 1.9 | 16.4 ± 2.7 | p = 0.22 |
| SSRI Antidepressant | Zoloft (2), Paxil | Zoloft (2), | p = 0.22 |
| | (2), Prozac (1), | Luvox (2), | |
| | Celexa (1) | Lexapro (1) | |
| Beck Depression Inventory | 20.5 ± 8.3 | 18.9 ± 10.6 | p = 0.79 |
| Hamilton Depression Score | 14.4 ± 2.3 | 11.9 ± 4.3 | p = 0.25 |
| 17 | | | |
| Hamilton Depression Score | 16.7 ± 2.2 | 14.8 ± 4.8 | p = 0.41 |
| 21 | | | |
| MADRS | 22.5 ± 5.6 | 22.2 ± 9.2 | p = 0.95 |

1. A method of treating a mood disorder in an individual suffering from the mood disorder, comprising administering an effective amount of an antagonist of high affinity nicotinic acetylcholine receptors and at least one agent for treatment of the mood disorder to the individual.

2. The method of claim 1 wherein the mood disorder is major depressive disorder, dysthymic disorder, depressive disorder NOS, or bipolar disorder.

3. The method of claim 2 wherein the mood disorder is major depressive disorder and the antagonist of high affinity nicotinic acetylcholine receptors is mecamylamine or a salt thereof.

4. The method of claim 3, wherein the agent for treatment of the mood disorder is a tricyclic antidepressant, a monoamine oxidase inhibitor, a selective serotonin reuptake inhibitor or a selective norepinephrine reuptake inhibitor, a norepinephrine dopamine reuptake inhibitor.

5. The method of claim 1 wherein the agent for treatment of a mood disorder is a selective serotonin reuptake inhibitor.

6. The method of claim 4 wherein the selective serotonin reuptake inhibitor is citalopram, clovoxamine, escitalopram, femoxetine, flesinoxan, fluoxetine, fluvoxamine, paroxetine, setraline, trazodone, or zimeldine.

7. The method of claim 5 wherein the selective serotonin reuptake inhibitor inhibitor is citalopram, clovoxamine, escitalopram, femoxetine, flesinoxan, fluoxetine, fluvoxamine, paroxetine, setraline, trazodone, or zimeldine.

8. The method of claim 4 wherein the treatment comprises administering mecamylamine and a selective serotonin uptake inhibitor in a single formulation.

9. A pharmaceutical formulation comprising an antagonist of high affinity nicotinic acetylcholine receptors and at least one agent for treatment of a mood disorder.

10. The pharmaceutical formulation of claim 9 wherein the antagonist of high affinity nicotinic acetylcholine receptors is mecamylamine.

11. The pharmaceutical formulation of claim 10 wherein the formulation additionally comprises a carrier or excipient.

12. The pharmaceutical formulation of claim 10 wherein the mecamylamine is mecamylamine hydrochloride.

13. The pharmaceutical formulation of claim 9 wherein the antagonist of high affinity nicotinic acetylcholine receptors is mecamylamine and the additional agent is a selective serotonin reuptake inhibitor.

14. The pharmaceutical formulation of claim 9 wherein the additional active agent is a tricyclic antidepressant, a monoamine oxidase inhibitor, a selective serotonin reuptake inhibitor, a selective norepinephrine reuptake inhibitor, or a norepinephrine dopamine reuptake inhibitor.

15. The pharmaceutical formulation of claim 14 wherein the additional agent is a selective serotonin reuptake inhibitor.

16. The pharmaceutical formulation of claim 13 wherein the selective serotonin reuptake inhibitor is citalopram, clovoxamine, escitalopram, femoxetine, flesinoxan, fluoxetine, fluvoxamine, paroxetine, sertraline, trazodone, or zimeldine.

17. The pharmaceutical formulation of claim 10 wherein the additional active agent is a selective norepinephrine reuptake inhibitor.

18. The pharmaceutical formulation of claim 9 which is for oral administration.

19. The pharmaceutical formulation of claim 18 which is a liquid, a syrup, a tablet, a capsule, a powder, a sprinkle, a chewtab, or a dissolvable disc.

20. The pharmaceutical formulation of claim 10 which is for oral administration.

21. The pharmaceutical formulation of claim 20 which is a liquid, a syrup, a tablet, a capsule, a powder, a sprinkle, a chewtab or a dissolvable disc.

22. The pharmaceutical formulation of claim 13 which is for oral administration.

23. The pharmaceutical formulation of claim 22 which is a liquid, a syrup, a tablet, a capsule, a powder, a sprinkle, a chewtab or a dissolvable disc.

24. A pharmaceutical formulation comprising an extended release form of mecamylamine and at least one additional active agent.
